(19) United States
(12) Patent Application Publication
(10) Pub. No.: US 2025/0109098 A1
(43) Pub. Date: Apr. 3, 2025

(54) METHOD FOR PREPARING BENZYLAMINE DERIVATIVE THROUGH AMINATION OF BENZYLIC C-H BOND UNDER VISIBLE LIGHT-INDUCED NICKEL CATALYSIS

(71) Applicant: SHANGHAI OURCHEM BIO-TECHNOLOGY CO., LTD., Shanghai (CN)

(72) Inventors: Shaofang ZHOU, Shanghai (CN); Congrui WU, Shanghai (CN); Fang LING, Shanghai (CN); Chunhua LIANG, Shanghai (CN)

(73) Assignee: SHANGHAI OURCHEM BIO-TECHNOLOGY CO., LTD., Shanghai (CN)

(21) Appl. No.: 18/848,143

(22) PCT Filed: Dec. 4, 2023
(86) PCT No.: PCT/CN2023/136030

§ 371 Date: Sep. 18, 2024

(30) Foreign Application Priority Data
Feb. 7, 2023 (CN) 202310069883.0

(51) Int. Cl. C07C 269/04; B01J 31/18; B01J 35/39; C07C 303/36

(52) U.S. Cl. CPC C07C 269/04; B01J 31/1825; B01J 35/39; C07C 303/36; B01J 2231/44; B01J 2531/004; B01J 2531/847; C07C 2601/16

(57) ABSTRACT

A method for preparing a benzylamine derivative through amination of a benzylic C—H bond under visible light-induced nickel catalysis is provided. The method includes: weighing and adding an amination reagent, a ruthenium or iridium photosensitizer, a nickel catalyst, a Lewis acid, and a ligand according to a molar ratio to a reaction vessel; under an inert gas atmosphere, adding a solvent, stirring, adding a benzyl-containing alkylate, and irradiating with a visible light source to allow a full reaction; and conducting separation and purification to obtain the benzylamine derivative. The method involves cheap and easily-available raw materials, and has relatively-extensive substrate applicability. In addition, the method has advantages such as mild reaction conditions, a high yield of a target product, small pollution, and a simple reaction operation and post-treatment, and thus is suitable for industrial production.

### CROSS REFERENCE TO THE RELATED APPLICATIONS

This application is the national phase entry of International Application No. PCT/CN2023/136030, filed on Dec. 4, 2023, which is based upon and claims priority to Chinese Patent Application No. 202310069883.0, filed on Feb. 7, 2023, the entire contents of which are incorporated herein by reference.

### TECHNICAL FIELD

The present disclosure belongs to the technical field of visible light-induced catalytic synthesis, and specifically relates to a method for preparing a benzylamine derivative through amination of a benzylic C—H bond under visible light-induced nickel catalysis.

### BACKGROUND

Compounds with benzylamine structures are an important class of organic compounds. There are similar structures in many biologically active compounds and natural products. The compounds with benzylamine structures are widely used particularly in medicine (Richter, M. F. Nature 545, 2017, 299-304). The physical and chemical properties and the biological activity of a natural product or bioactive molecule can be greatly changed by introducing nitrogen atoms into the natural product or bioactive molecule. For example, ampicillin, the first penicillin derivative with broad-spectrum antimicrobial activity, is produced by introducing a benzylamine structure into a structure of penicillin G. Ampicillin exhibits significantly improved antimicrobial activity and can inhibit both Gram-positive bacteria and Gram-negative bacteria (Acred, P.; Brown, D. M.; Turner, D. H.; Wilson, M. J. Br J. Pharmacol. 1962, 18, 356-369). In addition, many commercially available best-selling drugs approved by the Food and Drug Administration (FDA) include a benzylamine group, such as imatinib, clopidogrel, sertraline, and donepezil (McGrath, N. A.; Brichacek, M.; Njardarson, J. T. J. Chem. Educ. 2010, 87, 1348-1349). Therefore, the direct and selective introduction of a benzylamine group into complicated molecules can accelerate the discovery of bioactive molecules and further facilitate the exploration of drug candidates derived from natural products.

Traditionally, the introduction of a benzylamine structure is mainly conducted by the following process: a C—X bond (where X=oxygen, halogen, or the like) is pre-constructed to allow pre-functionalization, and then the transformation is conducted. However, this process is not suitable for the post-modification of some complicated molecules. It has been discovered that the transition metal-catalyzed nitrene-insertion reaction can be applied to an amination reaction for a series of C—H bonds. The precious metal Ru is a common catalyst, which can allow amination reactions of C—H bonds at benzyl, tertiary, and allyl positions under the regulation of a ligand. However, this catalyst is relatively expensive. In addition, when there are multiple active functional groups in a system, the chemoselectivity and the site selectivity are poor, and it is difficult to separate products. In recent years, the direct amination reaction of a C—H bond that is induced by a nitrene precursor and catalyzed by a cheap metal has also been developed rapidly. Cobalt, copper, iron, manganese, or the like are all used as catalysts to allow the synthesis of benzylamines, but a specific amination reagent or an excessive amount of an oxidant is generally required, resulting in failed general applicability and difficult extensive use.

The photocatalytic reaction has been a hot topic for scholars inside and outside China in recent years. The photocatalytic reaction can convert light energy into chemical energy to allow the chemical transformation through electron, atom, or energy transfer. The photocatalytic reaction can effectively avoid the use of an oxidant and an excessive amount of an alkali and can reduce the energy consumption and the generation of chemical waste. Therefore, the photocatalytic reaction has advantages such as mild reaction conditions, prominent atomic economy, and no pollution. Ming BAO and Xiaoqiang YU developed a method for photo/iron dual-catalytic amination of a benzylic C—H bond. However, this method is mainly suitable for benzyl amination of diphenylmethane compounds, and thus a scope of substrate application is greatly limited (Tang, J.-J.; Yu, X.; Wang, Y.; Yamamoto, Y.; Bao, M. Angew. Chem. Int. Ed. 2021, 60, 16426-16435).

In summary, the current methods for synthesizing benzylamines have many shortcomings, such as expensive catalysts, poor reaction selectivity, and low applicability. Therefore, it is very important to develop a synthesis method with mild reaction conditions, a wide application range, simple reaction steps, and simple raw materials.

### SUMMARY

In view of the problems existing in the prior art, a technical problem to be solved by the present disclosure is to provide a method for preparing a benzylamine derivative through amination of a benzylic C—H bond under visible light-induced nickel catalysis. The method involves cheap and easily-available raw materials, and has relatively-extensive substrate applicability. In addition, the method has advantages such as mild reaction conditions, a high yield of a target product, small pollution, and a simple reaction operation and post-treatment, and thus is suitable for industrial production.

To solve the above problems, the present disclosure adopts the following technical solutions:

A method for preparing a benzylamine derivative through amination of a benzylic C—H bond under visible light-induced nickel catalysis is provided, including: weighing and adding an amination reagent, a ruthenium or iridium photosensitizer, a nickel catalyst, a Lewis acid, and a ligand according to a molar ratio to a reaction vessel; under an inert gas atmosphere, adding a solvent, fully stirring, adding a benzyl-containing alkylate, and irradiating with a visible light source to allow a full reaction; and conducting separation and purification to obtain the benzylamine derivative. To determine whether the reaction is completed, thin layer chromatography (TLC), liquid chromatography (LC), gas chromatography (GC, when a molecular weight is less than 300), or the like can be adopted for monitoring.

The benzylamine derivative has the following general structural formula:

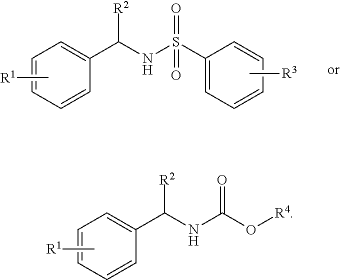

An equation of the reaction is as follows:

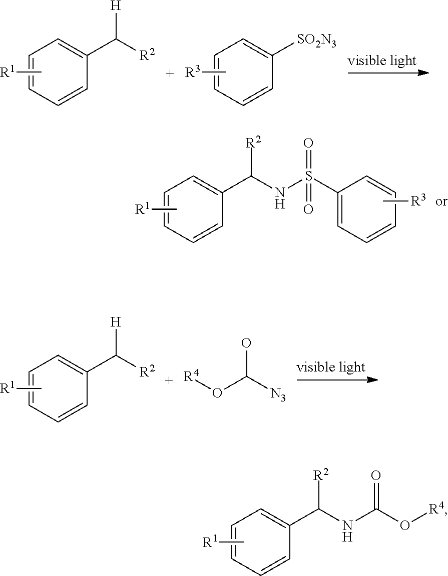

where R¹ is selected from any one of hydrogen, C₁-C₄ alkyl, alkoxy, aryl, and halogen;
R² is selected from any one of hydrogen, C₁-C₆ alkyl, and aryl; R³ is selected from any one of C₁-C₃ alkyl and halogen; and R⁴ is selected from any one of C₁-C₅ alkyl and aryl.

In the method for preparing a benzylamine derivative through amination of a benzylic C—H bond under visible light-induced nickel catalysis, a molar ratio of the benzyl-containing alkylate, the amination reagent, the ruthenium or iridium photosensitizer, the nickel catalyst, the Lewis acid, and the ligand is 1:(1-5):(0.05-0.5):(0.05-0.5):(1-3):(0.05-0.25); and an amount of the ligand is preferably 15% to 25% and further preferably 20% of an amount of the catalyst (that is, based on the benzyl-containing alkylate).

In the method for preparing a benzylamine derivative through amination of a benzylic C—H bond under visible light-induced nickel catalysis, a general structural formula of the benzyl-containing alkylate is

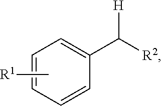

and a general structural formula of the amination reagent is

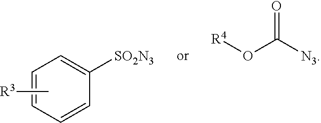

In the method for preparing a benzylamine derivative through amination of a benzylic C—H bond under visible light-induced nickel catalysis, a wavelength of the visible light source is 400 nm to 475 nm.

In the method for preparing a benzylamine derivative through amination of a benzylic C—H bond under visible light-induced nickel catalysis, the ruthenium or iridium photosensitizer is one of Ru(bpy)₃Cl₂, Ru(bpy)₃(PF₆)₂, Ir(ppy)₃, Ir(ppy)₂(dtbbpy)PF₆, and Ir[dF(CF₃)ppy)]₂(dtbbpy)PF₆.

Chemical structural formulas of these ruthenium or iridium photosensitizers are as follows:

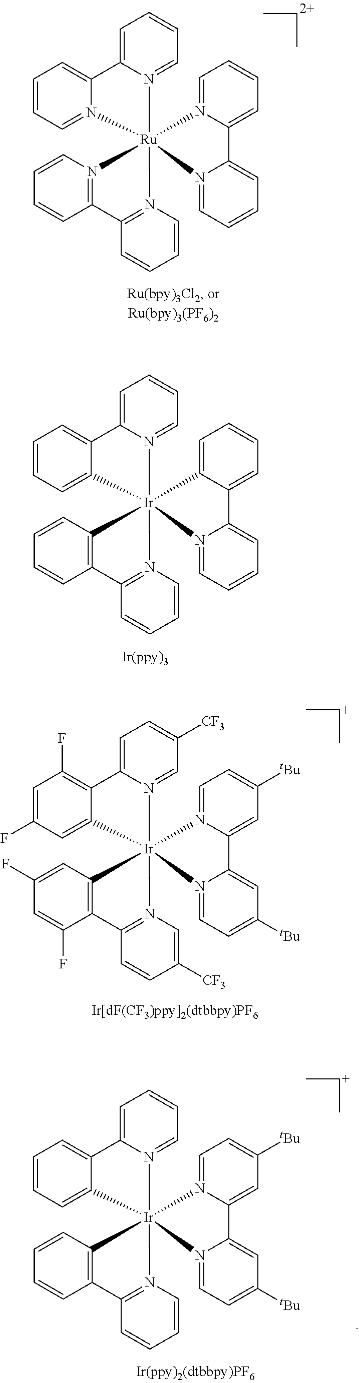

Ru(bpy)₃Cl₂ and Ru(bpy)₃(PF₆)₂ have the same metal moiety, but have different complex anions.

In the method for preparing a benzylamine derivative through amination of a benzylic C—H bond under visible light-induced nickel catalysis, the Lewis acid is one of fluoroboric acid or boron trifluoride etherate.

In the method for preparing a benzylamine derivative through amination of a benzylic C—H bond under visible light-induced nickel catalysis, the nickel catalyst is NiX₂ or NiX₂·dme, where X is one of Cl, Br, and I; and NiX₂·dme is preferably nickel(II) bromide ethylene glycol dimethyl ether complex (CAS: 28923-39-9).

In the method for preparing a benzylamine derivative through amination of a benzylic C—H bond under visible light-induced nickel catalysis, the ligand is one of a bipyridine ligand or a phenanthroline ligand.

Chemical structural formulas of these ligands are as follows:

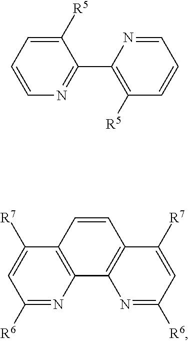

where R⁵, R⁷, and R¹ each are independently selected from one of hydrogen, methyl, tert-butyl, fluorine, chlorine, trifluoromethyl, cyano, and a formate group.

In the method for preparing a benzylamine derivative through amination of a benzylic C—H bond under visible light-induced nickel catalysis, the solvent is any one of acetonitrile, dichloromethane, 1,2-dichloroethane, tetrahydrofuran, or N,N-dimethylformamide; and an inert gas is nitrogen or argon.

In the method for preparing a benzylamine derivative through amination of a benzylic C—H bond under visible light-induced nickel catalysis, a product is purified by column chromatography or LC.

Beneficial effects: Compared with the prior art, the present disclosure has the following advantages:

1. The present disclosure adopts an alkyl-containing compound as a starting material, and this raw material is easy to obtain and has low toxicity, a low cost, and many types.

2. The amination reagent adopted in the present disclosure is cheap, easy to obtain, and convenient to use, and has low toxicity.

3. In the present disclosure, a reaction site is benzyl, the amination reagent is an azide, and there is a different product from the prior art. Thus, the method of the present disclosure is suitable not only for the synthesis of benzylsulfamide, but also for the synthesis of a benzyl ester amine.

4. The present disclosure involves mild reaction conditions, a short reaction time, a high yield of a target product, and a simple reaction operation and post-treatment, and thus is suitable for industrial production.

### DETAILED DESCRIPTION OF THE EMBODIMENTS

To make the objectives, features, and advantages of the present disclosure clear and comprehensible, specific implementations of the present disclosure will be described in detail below in conjunction with specific examples.

### Example 1

Synthesis of N-benzyl-p-toluenesulfonamide

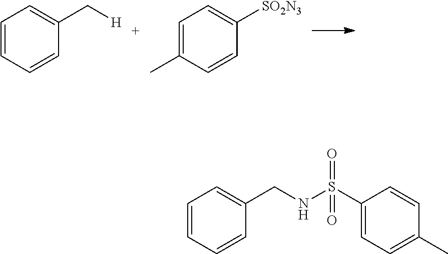

P-toluenesulfonyl azide and toluene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, toluene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 83%). Analytical data of the product was as follows: ¹H NMR (400 MHz, CDCl₃) δ 7.76 (d, J=7.9 Hz, 2H), 7.31 (d, J=7.9 Hz, 2H), 7.29-7.24 (m, 3H), 7.26-7.16 (m, 2H), 4.70 (brs, 1H), 4.12 (d, J=6.2 Hz, 2H), 2.44 (s, 3H); ¹³C NMR (101 MHz, CDCl₃) δ 143.69, 136.98, 136.39, 129.89, 128.84, 128.07, 128.01, 127.33, 47.42, 21.68.

### Example 2

### Synthesis of N-4-fluorobenzyl)p-toluenesulfonamide

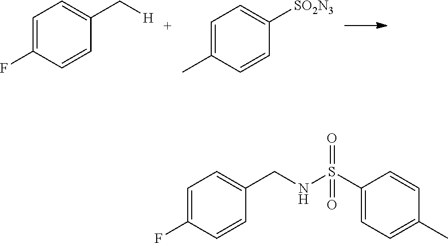

P-toluenesulfonyl azide and p-fluorotoluene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, p-fluorotoluene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 72%). Analytical data of the product was as follows: ¹H NMR (400 MHz, CDCl₃) δ 7.74 (d, J=8.0 Hz, 2H), 7.31 (d, J=8.0 Hz, 2H), 7.17 (dd, J=8.4, 5.4 Hz, 2H), 6.96 (t, J=8.5 Hz, 2H), 4.78 (t, J=6.4 Hz, 1H), 4.09 (d, J=6.2 Hz, 2H), 2.44 (s, 3H); ¹³C NMR (101 MHz, CDCl₃) δ 162.52 (d, J=246.7 Hz), 143.79, 136.97, 132.24 (d, J=3.6 Hz), 129.91, 129.76 (d, J=8.1 Hz), 127.29, 115.70 (d, J=21.7 Hz), 46.70, 21.68.

### Example 3

Synthesis of N-(4-chlorobenzyl)p-toluenesulfonamide

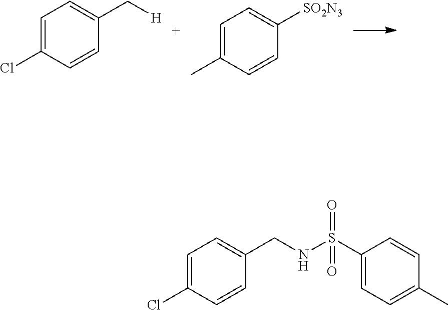

P-toluenesulfonyl azide and p-chlorotoluene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, p-chlorotoluene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 71%). Analytical data of the product was as follows: ¹H NMR (400 MHz, CDCl₃) δ 7.73 (d, J=8.1 Hz, 2H), 7.30 (d, J=8.1 Hz, 2H), 7.24 (d, J=8.5 Hz, 2H), 7.13 (d, J=8.5 Hz, 2H), 4.82 (t, J=6.4 Hz, 1H), 4.09 (d, J=6.3 Hz, 2H), 2.44 (s, 3H); ¹³C NMR (101 MHz, CDCl₃) δ 143.84, 136.92, 134.99, 133.90, 129.92, 129.35, 128.95, 127.28, 46.71, 21.68.

### Example 4

Synthesis of N-(4-bromobenzyl)p-toluenesulfonamide

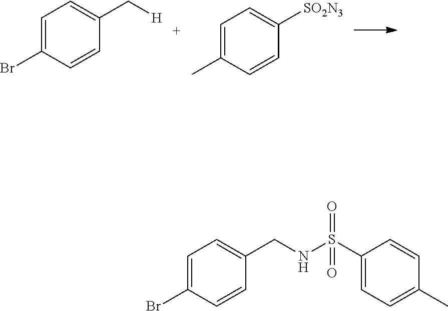

P-toluenesulfonyl azide and p-bromotoluene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, p-bromotoluene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 81%). Analytical data of the product was as follows: ¹H NMR (400 MHz, CDCl₃) δ 7.72 (d, J=8.1 Hz, 2H), 7.38 (d, J=8.4 Hz, 2H), 7.29 (d, J=8.1 Hz, 2H), 7.07 (d, J=8.4 Hz, 2H), 4.88 (t, J=6.3 Hz, 1H), 4.07 (d, J=6.4 Hz, 2H), 2.44 (s, 3H); ¹³C NMR (101 MHz, CDCl₃) δ 143.84, 136.91, 135.52, 131.89, 129.92, 129.67, 127.27, 121.97, 46.74, 21.69.

### Example 5

Synthesis of N-(2,4-difluorobenzyl)p-toluenesulfonamide

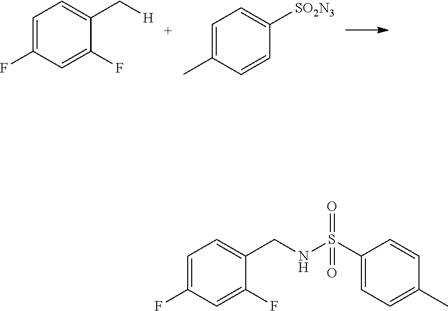

P-toluenesulfonyl azide and 2,4-difluorotoluene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, 2,4-difluorotoluene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 76%). Analytical data of the product was as follows: ¹H NMR (400 MHz, CDCl₃) δ 7.70 (d, J=8.1 Hz, 2H), 7.27 (d, J=8.1 Hz, 2H), 7.25-7.20 (m, 1H), 6.87-6.52 (m, 2H), 4.84 (t, J=6.5 Hz, 1H), 4.17 (d, J=6.5 Hz, 2H), 2.42 (s, 3H); ¹³C NMR (101 MHz, CDCl₃) δ 162.78 (dd, J=249.3, 11.7 Hz), 160.84 (dd, J=249.3, 11.7 Hz), 143.75, 137.00, 131.21 (dd, J=9.5, 5.7 Hz), 129.82, 127.20, 119.78 (dd, J=14.6, 3.7 Hz), 111.53 (dd, J=21.1, 3.6 Hz), 103.96 (t, J=25.4 Hz), 40.89 (d, J=3.5 Hz), 21.63.

### Example 6

Synthesis of N-(2-trifluoromethylbenzyl)p-toluenesulfonamide

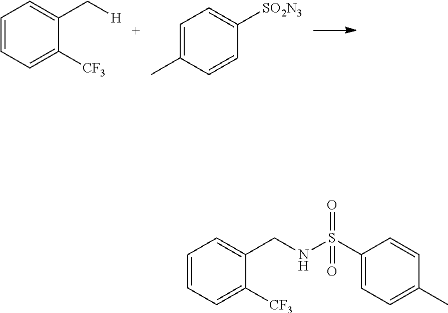

P-toluenesulfonyl azide and 2-methylbenzotrifluoride were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, 2-methylbenzotrifluoride (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 83%). Analytical data of the product was as follows: ¹H NMR (400 MHz, CDCl₃) δ 7.75 (d, J=8.4 Hz, 2H), 7.59 (t, J=7.8 Hz, 2H), 7.50 (t, J=8.1 Hz, 1H), 7.38 (t, J=7.6 Hz, 1H), 7.31 (d, J=7.9 Hz, 2H), 4.79 (t, J=6.6 Hz, 1H), 4.30 (d, J=6.6 Hz, 2H), 2.44 (s, 3H); ¹³C NMR (101 MHz, CDCl₃) δ 143.85, 136.97, 135.04, 132.50, 130.96, 129.94, 128.19 (q, J=30.5 Hz), 128.16, 127.24, 127.23, 126.13 (q, J=5.4 Hz), 124.33 (q, J=273.9 Hz), 43.89, 21.69.

### Example 7

Synthesis of N-(2-methylbenzyl)p-toluenesulfonamide

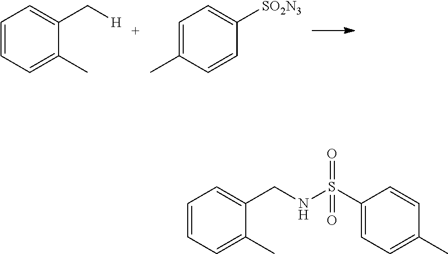

P-toluenesulfonyl azide and o-xylene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, o-xylene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 55%). Analytical data of the product was as follows: ¹H NMR (400 MHz, CDCl₃) δ 7.77 (d, J=7.9 Hz, 2H), 7.32 (d, J=7.9 Hz, 2H), 7.23-7.08 (m, 4H), 4.44 (t, J=6.0 Hz, 1H), 4.09 (d, J=5.9 Hz, 2H), 2.45 (s, 3H), 2.25 (s, 3H); ¹³C NMR (101 MHz, CDCl₃) δ 143.71, 136.89, 134.00, 130.78, 129.89, 129.01, 128.43, 127.36, 126.36, 45.58, 21.70, 18.93.

### Example 8

Synthesis of N-(4-methylbenzyl)p-toluenesulfonamide

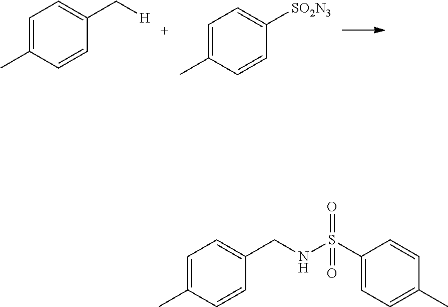

P-toluenesulfonyl azide and p-xylene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, p-xylene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 50%). Analytical data of the product was as follows: ¹H NMR (400 MHz, CDCl₃) δ 7.76 (d, J=7.9 Hz, 2H), 7.31 (d, J=7.9 Hz, 2H), 7.10-7.06 (m, 4H), 4.57 (t, J=6.1 Hz, 1H), 4.07 (d, J=6.0 Hz, 2H), 2.44 (s, 3H), 2.31 (s, 3H); ¹³C NMR (101 MHz, CDCl₃) δ 143.62, 137.85, 137.00, 133.33, 129.86, 129.50, 128.00, 127.34, 47.19, 21.67, 21.22.

### Example 9

Synthesis of N-(4-phenylbenzyl)p-toluenesulfonamide

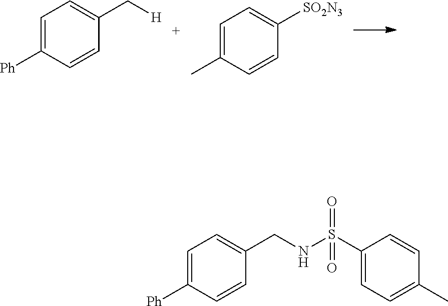

P-toluenesulfonyl azide and 4-methylbiphenyl were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, 4-methylbiphenyl (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 49%). Analytical data of the product was as follows: ¹H NMR (400 MHz, CDCl₃) δ 7.77 (d, J=7.9 Hz, 2H), 7.59 (d, J=7.8 Hz, 1H), 7.54 (d, J=7.6 Hz, 2H), 7.50 (d, J=7.9 Hz, 2H), 7.43 (t, J=7.7 Hz, 2H), 7.36 (d, J=7.3 Hz, 1H), 7.31 (d, J=8.1 Hz, 2H), 7.26 (d, J=7.3 Hz, 1H), 4.75 (brs, 1H), 4.17 (d, J=5.9 Hz, 2H), 2.43 (s, 3H); ¹³C NMR (101 MHz, CDCl₃) δ 143.71, 141.07, 140.64, 135.39, 129.90, 128.95, 128.47, 127.61, 127.55, 127.47, 127.35, 127.23, 127.18, 47.15, 21.68.

### Example 10

Synthesis of N-(4-tert-butylbenzyl)p-toluenesulfonamide

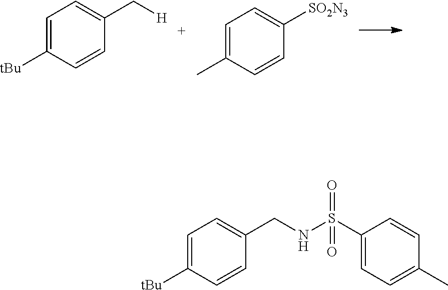

P-toluenesulfonyl azide and 4-tert-butyltoluene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, 4-tert-butyltoluene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 49%). Analytical data of the product was as follows: ¹H NMR (400 MHz, CDCl₃) δ 7.75 (d, J=7.9 Hz, 2H), 7.29 (d, J=7.9 Hz, 4H), 7.11 (d, J=7.9 Hz, 2H), 4.64 (t, J=6.6 Hz, 1H), 4.09 (d, J=6.0 Hz, 2H), 2.43 (s, 3H), 1.28 (s, 9H); ¹³C NMR (101 MHz, CDCl₃) δ 151.16, 143.56, 137.06, 133.33, 129.84, 127.80, 127.34, 125.74, 47.11, 34.66, 31.42, 21.67.

### Example 11

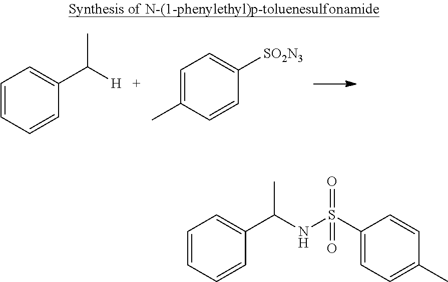

P-toluenesulfonyl azide and ethylbenzene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, ethylbenzene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 74%). Analytical data of the product was as follows: ¹H NMR (270 MHz, CDCl₃) δ 1.43 (d, 3H, J=7.0 Hz), 2.39 (s, 3H), 4.46 (dq, 1H, J=6.5, 7.0 Hz), 4.69 (d, 1H, J=6.5 Hz), 7.07-7.13 (m, 2H), 7.17-7.23 (m, 5H), 7.61 (dd, 2H, J=1.6, 6.8 Hz); ¹³C NMR (68 MHz, CDCl₃) δ 21.5, 23.5, 53.5, 125.9, 126.8, 127.3, 128.3, 129.2, 137.3, 141.7, 142.9.

### Example 12

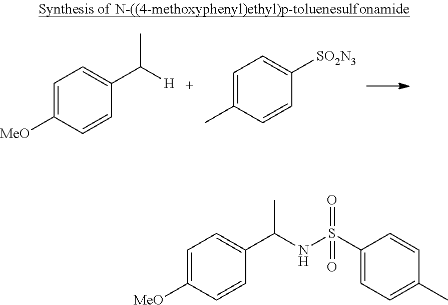

P-toluenesulfonyl azide and p-ethylanisole were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, p-ethylanisole (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 85%). Analytical data of the product was as follows: ¹H NMR (270 MHz, CDCl₃) δ 1.39 (d, 3H, J=7.0 Hz), 2.39 (s, 3H), 3.74 (s, 3H), 4.40 (dq, 1H, J=7.0 Hz), 5.06 (d, 1H, J=7.0 Hz), 6.70 (dd, 2H, J=2.2, 9.5 Hz), 7.00 (dd, 2H, J=2.2, 9.5 Hz), 7.18 (d, 2H, J=8.4 Hz), 7.61 (d, 2H, J=8.4 Hz); ¹³C NMR (68 MHz, CDCl₃) δ 21.5, 23.5, 53.1, 55.2, 113.7, 127.0, 127.2, 129.3, 134.0, 137.5, 142.8, 158.6.

### Example 13

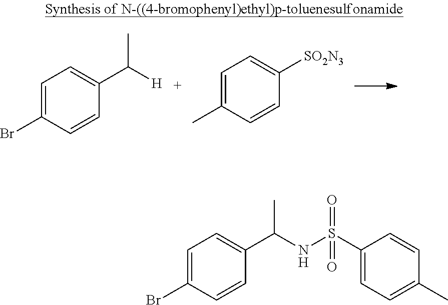

P-toluenesulfonyl azide and p-bromoethylbenzene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, p-bromoethylbenzene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 61%). Analytical data of the product was as follows: ¹H NMR (270 MHz, CDCl₃) δ 1.37 (d, 3H, J=6.8 Hz), 2.40 (s, 3H), 4.41 (dq, 1H, J=7.0, 6.8 Hz), 5.37 (d, 1H, J=7.0 Hz), 6.96 (d, 2H, J=8.6 Hz), 7.16 (d, 2H, J=8.1 Hz), 7.26 (d, 2H, J=8.1 Hz), 7.57 (d, 2H, J=8.6 Hz); ¹³C NMR (68 MHz, CDCl₃) δ 21.5, 23.4, 53.1, 121.0, 126.9, 127.8, 129.3, 131.3, 137.2, 140.9, 143.2.

### Example 14

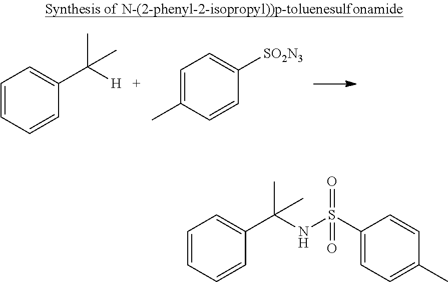

P-toluenesulfonyl azide and cumene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, cumene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 70%). Analytical data of the product was as follows: ¹H NMR (270 MHz, CDCl₃) δ 1.61 (s, 6H), 2.37 (s, 3H), 5.38 (s, 1H), 7.12-7.19 (m, 5H), 7.25-7.31 (m, 2H), 7.56 (d, 2H, J=8.6 Hz); ¹³C NMR (68 MHz, CDCl₃) δ 21.5, 29.8, 58.5, 125.4, 126.8 (2C), 127.9, 129.1, 139.6, 142.4, 145.0.

### Example 15

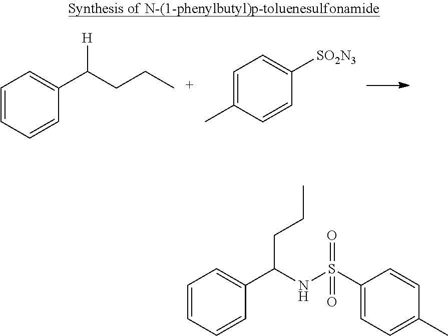

P-toluenesulfonyl azide and butylbenzene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, butylbenzene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 73%). Analytical data of the product was as follows: ¹H NMR (270 MHz, CDCl₃) δ 0.82 (t, 3H, J=7.3 Hz), 1.12-1.28 (m, 2H), 1.62-1.77 (m, 2H), 2.34 (s, 3H), 4.27 (dt, 1H, J=7.3 Hz), 5.06 (d, 1H, J=7.3 Hz), 6.98-7.02 (m, 2H), 7.08-7.15 (m, 5H), 7.53 (d, 2H, J=8.4 Hz); ¹³C NMR (68 MHz, CDCl₃) δ 13.5, 19.1, 21.4, 39.7, 58.1, 126.5, 127.0, 127.2, 128.3, 129.2, 137.7, 141.0, 142.8.

### Example 16

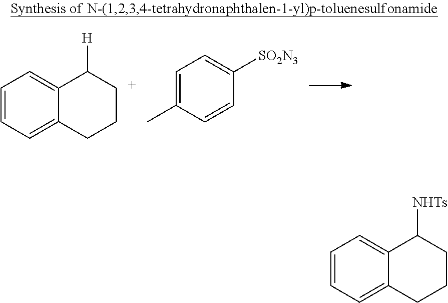

P-toluenesulfonyl azide and tetrahydronaphthalene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, tetrahydronaphthalene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 76%). Analytical data of the product was as follows: ¹H NMR (270 MHz, CDCl₃) δ 1.69-1.86 (m, 4H), 2.46 (s, 3H), 2.57-2.81 (m, 2H), 4.41-4.47 (m, 1H), 4.69 (d, 1H, J=7.6 Hz), 6.93 (d, 11H, J=7.6 Hz), 7.02-7.16 (m, 3H), 7.34 (d, 2H, J=8.6 Hz), 7.82 (d, 2H, J=8.6 Hz); ¹³C NMR (68 MHz, CDCl₃) δ 19.2, 21.7, 28.9, 30.8, 51.9, 126.2, 127.0, 127.5, 128.7, 129.1, 129.6, 135.4, 137.4, 137.9, 143.2.

### Example 17

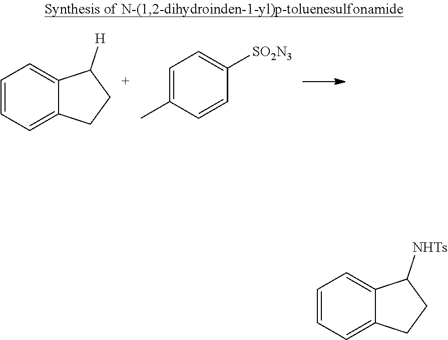

P-toluenesulfonyl azide and dihydroindene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, dihydroindene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 88%). Analytical data of the product was as follows: ¹H NMR (270 MHz, CDCl₃) δ 1.71-1.82 (m, 1H), 2.29-2.40 (m, 1H), 2.46 (s, 3H), 2.71-2.80 (m, 1H), 2.85-2.91 (m, 1H), 4.66 (d, 1H, J=8.6 Hz), 4.82 (dt, 1H, J=8.6, 7.3 Hz), 7.06-7.21 (m, 4H), 7.34 (d, 2H, J=8.6 Hz), 7.84 (d, 2H, J=8.6 Hz); ¹³C NMR (68 MHz, CDCl₃) δ 21.6, 30.0, 34.7, 58.7, 124.0, 124.7, 126.7, 127.0, 128.1, 129.7, 138.0, 141.8, 142.7, 143.3.

### Example 18

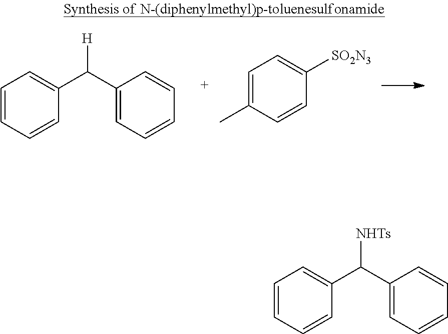

P-toluenesulfonyl azide and diphenylmethane were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, diphenylmethane (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 96%). Analytical data of the product was as follows: ¹H NMR (270 MHz, CDCl₃) δ 2.36 (s, 3H), 5.25 (br s, 1H), 5.56 (d, 1H, J=7.3 Hz), 6.81-7.49 (m, 12H), 7.55 (d, 2H, J=6.5 Hz); ¹³C NMR (68 MHz, CDCl₃) δ 21.5, 61.4, 127.1, 127.3, 127.4, 128.4, 129.2, 137.3, 140.4, 143.0.

### Example 19

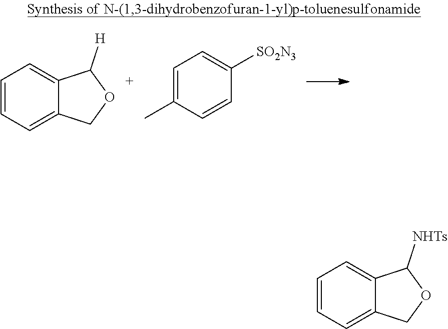

P-toluenesulfonyl azide and 1,3-dihydrobenzofuran were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, 1,3-dihydrobenzofuran (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 88%). Analytical data of the product was as follows: ¹HNMR (270 MHz, CDCl₃) δ 2.45 (s, 3H), 4.91 (d, 1H, J=12.7 Hz), 5.00 (d, 1H, J=12.7 Hz), 5.21 (d, 1H, J=10.3 Hz), 6.54 (d, 1H, J=10.3 Hz), 7.19-7.39 (m, 6H), 7.86 (d, 2H, J=8.4 Hz). ¹³C NMR (68 MHz, CDCl₃) δ 21.6, 72.0, 88.9, 121.0, 122.9, 127.1, 128.0, 129.4, 129.5, 136.5, 138.5, 139.1, 143.3.

### Example 20

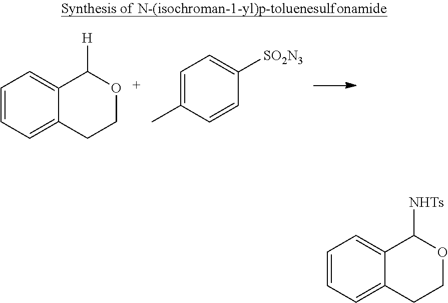

P-toluenesulfonyl azide and 1,3-dihydrobenzofuran were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-toluenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, 1,3-dihydrobenzofuran (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 88%). Analytical data of the product was as follows: ¹HNMR (270 MHz, CDCl₃) δ 2.45 (s, 3H), 4.91 (d, 1H, J=12.7 Hz), 5.00 (d, 1H, J=12.7 Hz), 5.21 (d, 1H, J=10.3 Hz), 6.54 (d, 1H, J=10.3 Hz), 7.19-7.39 (m, 6H), 7.86 (d, 21, J=8.4 Hz); ¹³C NMR (68 MHz, CDCl₃) δ 21.6, 72.0, 88.9, 121.0, 122.9, 127.1, 128.0, 129.4, 129.5, 136.5, 138.5, 139.1, 143.3.

### Example 21

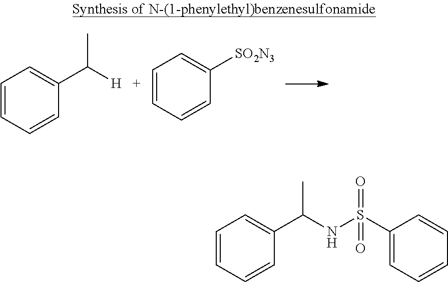

Benzenesulfonyl azide and ethylbenzene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and benzenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, ethylbenzene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 66%). Analytical data of the product was as follows: ¹HNMR (600 MHz, CDCl₃): δ 7.73 (d, J=7.6 Hz, 2H), 7.51-7.47 (m, 1H), 7.40-7.36 (m, 2H), 7.21-7.16 (m, 3H), 7.11-7.06 (m, 2H), 5.05 (d, J=7.1 Hz, 1H), 4.53-4.47 (m, 1H), 1.44 (d, J=6.8 Hz, 3H); ¹³CNMR (150 MHz, CDCl₃): δ 141.7, 140.5, 132.3, 128.8, 128.5, 127.5, 127.0, S6 126.0, 53.7, 23.6.

### Example 22

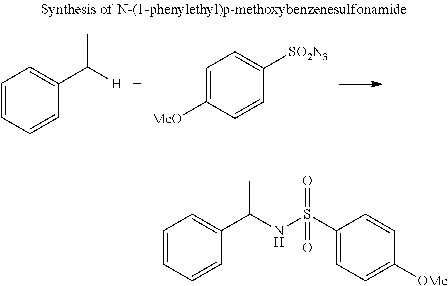

P-methoxybenzenesulfonyl azide and ethylbenzene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-methoxybenzenesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, ethylbenzene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 66%). Analytical data of the product was as follows: ¹HNMR (600 MHz, CDCl₃): δ 7.66 (d, J=8.8 Hz, 2H), 7.22-7.16 (m, 3H), 7.13-7.09 (m, 2H), 6.84 (d, J=8.8 Hz, 2H), 5.24 (d, J=7.1 Hz, 1H), 4.49-4.40 (m, 1H), 3.83 (s, 3H), 1.42 (d, J=6.8 Hz, 3H); ¹³CNMR (150 MHz, CDCl₃): δ 162.5, 142.0, 132.1, 129.1, 128.4, 127.3, 126.1, 113.9, 55.5, 53.6, 23.6.

### Example 23

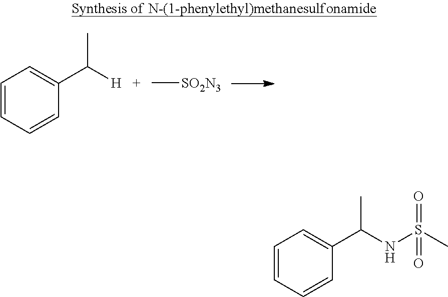

P-methanesulfonyl azide and ethylbenzene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-methanesulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, ethylbenzene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 45%). Analytical data of the product was as follows: ¹HNMR (600 MHz, CDCl₃): δ 7.40-7.33 (m, 4H), 7.33-7.29 (m, 1H), 4.98 (d, J=6.3 Hz, 1H), 4.69-4.60 (m, 1H), 2.62 (s, 3H), 1.55 (d, J=7.1 Hz, 3H); BC NMR (150 MHz, CDCl₃): δ 142.3, 128.9, 128.0, 126.2, 53.7, 41.7, 24.0.

### Example 24

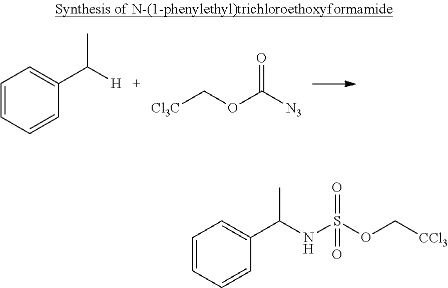

P-trichloroethoxyformyl azide and ethylbenzene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-trichloroethoxyformyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, ethylbenzene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 45%). Analytical data of the product was as follows: ¹H NMR (600 MHz, CDCl₃): δ 7.29 (d, J=8.5 Hz, 2H), 6.91 (d, J=8.5 Hz, 2H), 4.96 (d, J=6.8 Hz, 1H), 4.73-4.67 (m, 1H), 4.46 (d, J=10.8 Hz, 1H), 4.43 (d, J=10.8 Hz, 1H), 3.81 (s, 3H), 1.62 (d, J=6.8 Hz, 3H); ¹³C NMR (150 MHz, CDCl₃): δ 159.4, 133.3, 127.5, 114.3, 93.3, 78.0, 55.3, 54.4, 22.6.

### Example 25

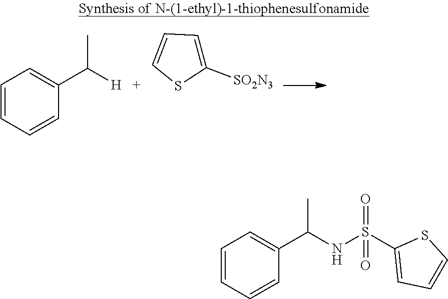

P-trichloroethoxysulfonyl azide and ethylbenzene were adopted as raw materials. Reaction steps were as follows:

(1) Tris(2-phenylpyridine)iridium (Ir(ppy)₃, 0.004 mmol), nickel dibromide (NiBr₂·dme, 0.04 mmol), 2,9-dimethyl-1,10-phenanthroline (0.048 mmol), and p-trichloroethoxysulfonyl azide (0.4 mmol) were added to a reaction flask, and the reaction flask was evacuated and subjected to gas replacement three times to make the reaction flask in an inert gas atmosphere. Under the protection of the inert gas atmosphere, acetonitrile (1 mL) was added, boron trifluoride etherate (0.2 mmol) was added dropwise, stirring was conducted for 5 min to allow thorough mixing, ethylbenzene (0.2 mmol) was added, and irradiation was conducted with a 475 nm blue LED lamp to allow a reaction at room temperature for 24 h.

(2) The reaction was monitored by TLC until the reaction was completed.

(3) A crude product obtained after the reaction was completed was separated through column chromatography (ethyl acetate:petroleum ether=1:20) to obtain a target product (yield: 54%). Analytical data of the product was as follows: ¹HNMR (600 MHz, CDCl₃): δ 7.49 (d, J=4.9 Hz, 1H), 7.42 (d, J=3.7 Hz, 1H), 7.25-7.18 (m, 3H), 7.15 (d, J=7.6 Hz, 2H), 6.94 (t, J=4.3 Hz, 1H), 5.30 (d, J=6.8 Hz, 1H), 4.60-4.52 (m, 1H), 1.48 (d, J=6.8 Hz, 3H); ¹³CNMR (150 MHz, CDCl₃): δ 141.7, 141.6, 132.2, 131.7, 128.5, 127.5, 127.1, 126.0, 54.0, 23.5.

What is claimed is:

1. A method for preparing a benzylamine derivative through amination of a benzylic C—H bond under a visible light-induced nickel catalysis, comprising: weighing and adding an amination reagent, a ruthenium or iridium photosensitizer, a nickel catalyst, a Lewis acid, and a ligand according to a molar ratio to a reaction vessel; under an inert gas atmosphere, adding a solvent to the reaction vessel, fully stirring, adding a benzyl-containing alkylate to the reaction vessel, and irradiating with a visible light source to allow a full reaction; and conducting separation and purification to obtain the benzylamine derivative with the following general structural formula: 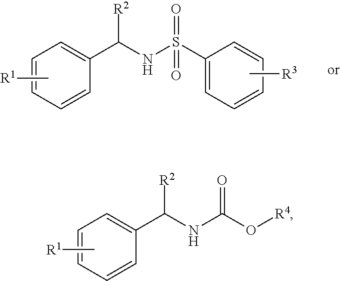
wherein R¹ is selected from one of hydrogen, C₁-C₄ alkyl, alkoxy, aryl, and halogen;
R² is selected from one of hydrogen, C₁-C₆ alkyl, and aryl; R³ is selected from one of C₁-C₃ alkyl and halogen; and R⁴ is selected from one of C₁-C₅ alkyl and aryl.

2. The method for preparing the benzylamine derivative through the amination of the benzylic C—H bond under the visible light-induced nickel catalysis according to claim 1, wherein a molar ratio of the benzyl-containing alkylate, the amination reagent, the ruthenium or iridium photosensitizer, the nickel catalyst, the Lewis acid, and the ligand is 1:(1-5):(0.05-0.5):(0.05-0.5):(1-3):(0.05-0.25).

3. The method for preparing the benzylamine derivative through the amination of the benzylic C—H bond under the visible light-induced nickel catalysis according to claim 1, wherein a general structural formula of the benzyl-containing alkylate is 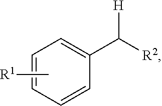
and a general structural formula of the amination reagent is

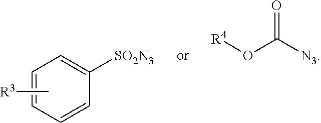

4. The method for preparing the benzylamine derivative through the amination of the benzylic C—H bond under the visible light-induced nickel catalysis according to claim 1, wherein a wavelength of the visible light source is 400 nm to 475 nm.

5. The method for preparing the benzylamine derivative through the amination of the benzylic C—H bond under the visible light-induced nickel catalysis according to claim 1, wherein the ruthenium or iridium photosensitizer is one of Ru(bpy)₃Cl₂, Ru(bpy)₃(PF₆)₂, Ir(ppy)₃, Ir(ppy)₂(dtbbpy)PF₆, and Ir[dF(CF₃)ppy)]₂(dtbbpy)PF₆.

6. The method for preparing the benzylamine derivative through the amination of the benzylic C—H bond under the visible light-induced nickel catalysis according to claim 1, wherein the Lewis acid is one of fluoroboric acid or boron trifluoride etherate.

7. The method for preparing the benzylamine derivative through the amination of the benzylic C—H bond under the visible light-induced nickel catalysis according to claim 1, wherein the nickel catalyst is NiX₂ or NiX₂·dme, wherein X is one of Cl, Br, and I.

8. The method for preparing the benzylamine derivative through the amination of the benzylic C—H bond under the visible light-induced nickel catalysis according to claim 1, wherein the ligand is one of a bipyridine ligand or a phenanthroline ligand.

9. The method for preparing the benzylamine derivative through the amination of the benzylic C—H bond under the visible light-induced nickel catalysis according to claim 1, wherein the solvent is one of acetonitrile, dichloromethane, 1,2-dichloroethane, tetrahydrofuran, or N,N-dimethylformamide; and an inert gas is nitrogen or argon.

10. The method for preparing the benzylamine derivative through the amination of the benzylic C—H bond under the visible light-induced nickel catalysis according to claim 1, wherein a product is purified by column chromatography or liquid chromatography (LC).
